(19) United States
(12) Patent Application Publication
(10) Pub. No.: US 2024/0182427 A1
(43) Pub. Date: Jun. 6, 2024

(54) SOLID DOSAGE FORMS OF A PLASMA KALLIKREIN INHIBITOR

(71) Applicant: Rezolute, Inc., Redwood City, CA (US)

(72) Inventors: Michael Burke, Bend, OR (US); Jeffrey Breit, Bend, OR (US); Randy Wald, Bend, OR (US); William Steinhauff, Bend, OR (US)

(21) Appl. No.: 18/516,315

(22) Filed: Nov. 21, 2023

Related U.S. Application Data
(60) Provisional application No. 63/384,641, filed on Nov. 22, 2022.

(51) Int. Cl. C07D 231/14; A61K 9/20

(52) U.S. Cl. CPC C07D 231/14; A61K 9/2009; A61K 9/2018; A61K 9/2027; A61K 9/2054; A61K 9/0053; C07B 2200/13

(57) ABSTRACT

Described herein are high loading, stable solid dosage forms of a plasma kallikrein inhibitor, 1-benzyl-1H-pyrazole-4-carboxylic acid 4-carbamimidoylbenzylamide (Compound 1) or a pharmaceutically acceptable salt thereof. Methods of treatment of patients that will benefit from the effects of a plasma kallikrein inhibitor are also disclosed.

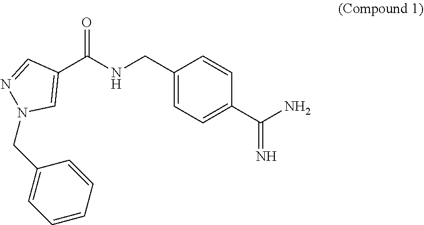 [SMILES: N=C(N)c1ccc(CNC(=O)c2cnn(Cc3ccccc3)c2)cc1]

### CROSS-REFERENCES TO RELATED APPLICATIONS

This application claims priority to U.S. Provisional Application No. 63/384,641, filed Nov. 22, 2022, which is incorporated herein in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Plasma kallikrein (PK) is a serine protease synthesized mainly in the liver from the proenzyme, prekallikrein. The proteolytic processing of prekallikrein plays a vital role in maintaining good health and is implicated in a wide range disease states. For example, in hereditary angioedema (HAE), there is a deficiency C1, an endogenous inhibitor of plasma kallikrein, and consequently this leads to bradykinin-mediated edema. HAE and other disease states that arise from high vascular permeability such as diabetes-induced retinal vascular permeability (e.g. Diabetic Macular Edema or DME) can be modulated by replacement of C1 or by administering a plasma kallikrein inhibitor (PKi).

A large number of small-molecule PKis have been disclosed, several are in clinical development, and one is approved. See, for example, U.S. Pat. Nos. 7,625,944; 8,258,170; 9,533,987; 9,738,641; 9,834,513; 10,759,759; 10,125,102; 10,221,161; 10,364,238; 10,562,850; 11,180,484. Patients in need of PKis can be treated by delivery via a variety of routes. One preferred route is delivery of the PKi orally to the patient. In order for such treatment to be commercially successful, the PKi must be manufactured in a pure, stable and orally bioavailable form that is then delivered to the patient in the correct amount and frequency. Delivery of the PKi as a solid dosage form that is ingested orally is a particularly desirable and convenient method. Although there is a large body of knowledge related to the formulation and preparation of oral solid dosage forms and there are many disclosures of the composition and preparation of solid dosage forms of specific compounds, the specific formulation that will have the desired properties varies with the specific compound and form to be delivered.

1-benzyl-1H-pyrazole-4-carboxylic acid 4-carbamimidoylbenzylamide (Compound 1) is useful as a plasma kallikrein inhibitor (PKi) for the prevention and treatment of PK dependent diseases. The non-clinical in vivo properties of Compound 1, also known as RZ402, has been described in “Nonclinical safety and pharmacology of RZ402, a plasma kallikrein inhibitor, for the treatment of diabetic macular edema as a daily oral therapy”; ARVO Annual Meeting Abstract, June 2020. Compound 1 is useful as a PKi, for the prevention and treatment of plasma kallikrein-dependent diseases or conditions, including disorders of blood coagulation, such as thrombosis, and other PK-dependent diseases and conditions. For example, the compounds inhibit the formation of thrombin by the intrinsic pathway and thus reduce the risk of new pathogenic thrombus formation (reocclusion), and also improve fibrinolytic induced reperfusion when given as adjunctive therapy with a fibrinolytic regimen. Compound 1 is also useful for treating other disease and disorders that are mediated by plasma kallikrein, for example, without limitation, diabetic macular edema, diabetic retinopathy, hereditary angioedema with inhibitor deficiency, acute liver injury, inflammation and anaphylaxis, exacerbation of hemorrhagic transformation and cerebral edema after treatment with recombinant tissue plasminogen activator (DPA), chemical-sensitized renal damage, ischemic stroke, hemorrhagic stroke, hypertension and its vascular complications (including retinopathy and nephropathy), cerebrovascular edema, pulmonary hypertension, inflammation, pain, acute myocardial infarction (MI), deep vein thrombosis (DVT), complications from fibrinolytic treatment (e.g., with tissue plasminogen activator, streptokinase following stroke or MI, angina, angioedema, sepsis, arthritis, complications of cardiopulmonary bypass, capillary leak syndrome, inflammatory bowel disease, diabetes and its vascular complications (including retinopathy, diabetic macular edema, nephropathy and neuropathy), age-related macular degeneration, retinal vein occlusions, brain edema, ischemia—reperfusion injury, angiogenesis (e.g., in cancer), asthma, anaphylaxis, and cerebrovascular complications of neurological conditions (e.g., Alzheimer's Disease, Parkinson's Disease, multiple sclerosis, CNS infections, and glioblastoma multiforme).

Compound 1 was initially disclosed in application Ser. No. 11/830,539, filed on Jul. 30, 2007, published as US 2008/0038276 A1 on Feb. 14, 2008, now U.S. Pat. No. 7,625,944. Despite the disclosure of this compound, improved pharmaceutical formulations that provide the desired stability, manufacturability, dissolution, pharmacokinetics and small size remain elusive.

As such, there exists a need to produce pharmaceutical formulations suitable for administration to humans or other animals. The present disclosure addresses these needs and provides related advantages as well.

### BRIEF SUMMARY OF THE INVENTION

In some aspects, provided herein are pharmaceutical compositions for oral delivery comprising:
a) 1-benzyl-1H-pyrazole-4-carboxylic acid 4-carbamimidoylbenzylamide (Compound 1)

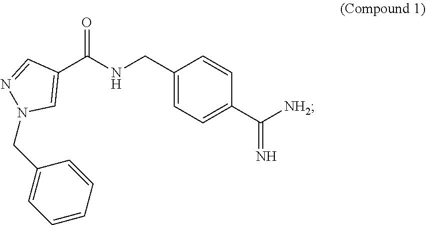 [SMILES: N=C(N)c1ccc(CNC(=O)c2cnn(Cc3ccccc3)c2)cc1]

b) a flow aid;
c) a disintegrant; and
d) a lubricant.

In some embodiments, the pharmaceutical composition further comprises,
e) a brittle filler,
f) a ductile filler and
g) an anti-adherent.

In some aspects, provided herein is a solid dosage form for oral delivery comprising
a) 1-benzyl-1H-pyrazole-4-carboxylic acid 4-carbamimidoylbenzylamide (Compound 1)

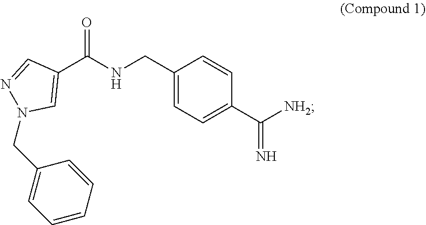 [SMILES: N=C(N)c1ccc(CNC(=O)c2cnn(Cc3ccccc3)c2)cc1]

b) a flow aid;
c) a disintegrant; and
d) a lubricant.

In some embodiments, the solid dosage form for oral delivery further comprises,
e) a brittle filler,
f) a ductile filler and
g) an anti-adherent.

In some embodiments, the solid dosage form is a tablet.

In some embodiments, provided herein is a kit comprising a pharmaceutical composition or a solid dosage form described herein.

In some embodiments, provided herein are methods of treating a plasma kallikrein-dependent condition or disease comprising orally delivering the formulations, compositions, or dosage forms described herein.

Other objects, features, and advantages of the present disclosure will be apparent to one of skill in the art from the following detailed description and figures.

### BRIEF DESCRIPTION OF THE DRAWINGS

FIG. 1 shows exemplary dissolution profiles of encapsulated powders comprising Compound 1 as described in Example 3 (open triangles) as compared to tablet formulations of Compound 1 as described in Example 2 (open circles and squares).

FIG. 2 shows exemplary dissolution profiles of encapsulated powders comprising Compound 1 as described in Example 3 (open triangle) as compared to tablet formulations of Compound 1 as described in Example 2 (open circles).

FIG. 3 shows exemplary dissolution profiles of tablet formulations of Compound 1 as described in Example 2 (open circles) as compared to the same tablet formulation tested after aging for 4 weeks at 40° C. and 75% RH (open squares).

FIG. 4 shows two different sized tablet punches used to prepare tablet compactions of the currently described pharmaceutical compositions

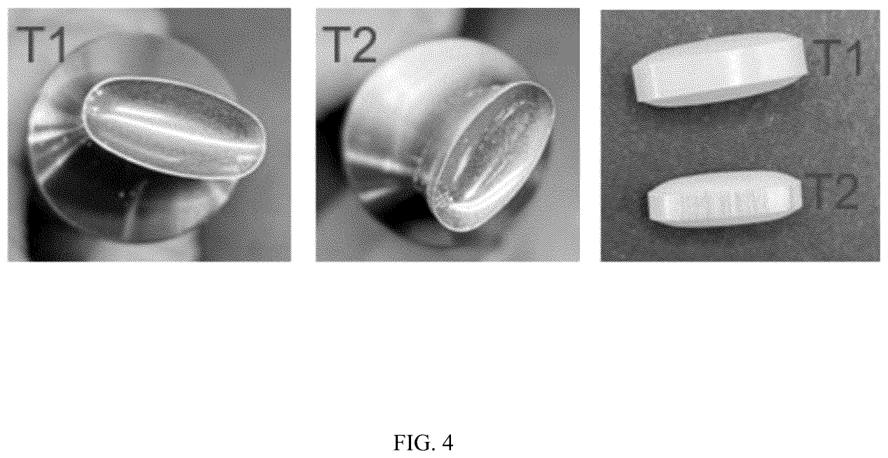

### DETAILED DESCRIPTION OF THE INVENTION

### I. General

During the development of Compound 1 for use in therapy, Applicant has discovered that Compound 1 has a variety of properties that make its delivery challenging. Specifically, Compound 1 in crystalline form such as crystalline acetate or chloride salts have poor flow properties requiring mitigation by formulation with various excipients or special processing techniques. Compound 1 is also chemically unstable when stored in the presence of formulation excipients—particularly those preferred for mitigating poor flow. In addition, Compound 1 has low intestinal permeability and though its solubility is high (that is, it is classified as a “BCS Class 3 drug”) it is preferable for its dissolution upon ingestion to be rapid to promote maximum absorption of Compound 1 into systemic circulation.

Disclosed herein are Formulations, dosage forms and methods of treatment of patients with such dosage forms. The formulations comprise Compound 1, a disintegrant, a flow aid and a lubricant. Optionally, the formulations may also comprise one or more fillers. Such fillers comprise brittle fillers and ductile fillers. In addition, the formulations may also optionally comprise an anti-adherent. In describing the invention, the inventors describe the class of materials that comprise the formulations and dosage forms and give examples of such materials. The dosage forms are oral dosage forms such as compressed solid forms comprising said formulations. Methods of treating a plasma kallikrein dependent diseases or conditions in a subject comprise orally administering to said subject said formulations or said dosage forms.

The formulations disclosed herein have improved physical stability. That is, the physical form and properties of the formulation have very little change over time. When the form of Compound 1 is an amorphous form, this means that the drug form remains substantially amorphous. When the form of Compound 1 is a crystalline form, this means that the drug substantially remains in the crystalline form used in the manufacture of the formulation. Improved physical stability is also reflected, and can be measured, in terms of dissolution in a use environment such as an in vitro dissolution test or the in vivo GI tract. In general, the formulations of the present invention retain their rapid dissolution properties over time.

The formulations of the present disclosure are chemically stable relative to other formulations of Compound 1. In general formulations of the present invention show a reduced increase in the amount of degradants present over time upon storage. In assessing chemical stability, the rate of increase of 1) total degradants or 2) the “RZ402 amide impurity” can be measured or 3) the rate of decrease in Compound 1 can be measured. Without being bound to any particular theory, it is believed that the amount of degradant is formed by hydrolysis and loss of ammonia. Such hydrolysis reactions can be base or acid catalyzed. It is believed that the presence of higher amounts of a flow aid such as silicon dioxide increases the rate of formation of the “Amide Degradant.”.

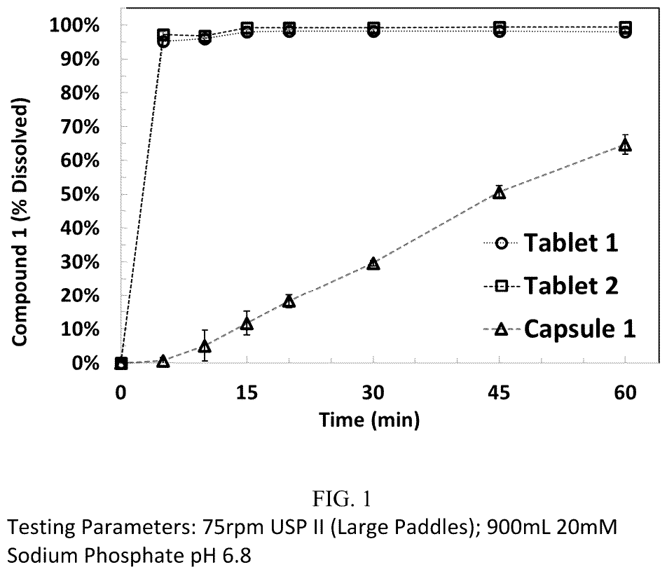

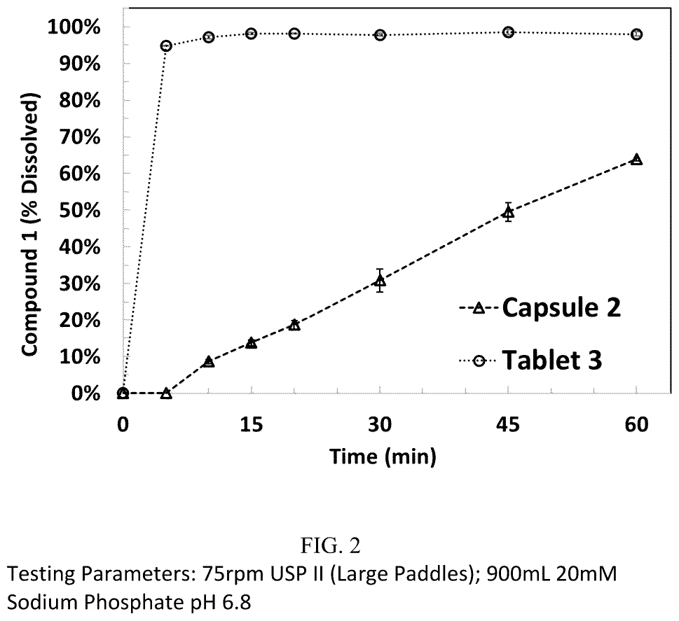

It is desirable for the formulations and dosage forms of the present invention to have a high drug loading to make the amount of material dosed acceptably small but low enough to achieve acceptably easy manufacture and acceptable dissolution and in vivo performance. Thus, the formulations & dosage forms of the present invention have drug loadings of greater than about 25 wt %. The inventors of the present invention have found that at high loadings of Compound 1 up to 50%, the dosage forms disintegrate and dissolve maintaining a rapid dissolution rate. (See, for example, FIGS. 1 and 2) However, high drug loading in the dosage form can lead to challenging manufacturing issues such as decreased flow, sticking to punches used to form compressed tablets and “banding—that is separation of the tablet material into distinct horizontal layers rather than a homogenous mixture Thus, it is also desirable that the amount of Compound 1 in the formulation or dosage form, is not too high. Accordingly, compositions and dosage forms of the present invention are comprised of less than about 70 wt % Compound 1. In some embodiments, the compositions and dosage forms of the present invention comprise more than about 30% Compound 1 but less than about 50% Compound 1.

The formulations of the present invention have sufficient flowability to be acceptably easy manufacture. In this context, flow can be measured by determining the bulk and tap density and using the industry standard Carr Index calculation. Additionally manual sifting of intra and extra granular material can be performed.

The dosage forms of the present invention disintegrate and dissolve to deliver Compound 1 to a use environment such that the target Compound 1 concentration in the use environment over time is achieved. Dissolution of the dosage forms can be measured by a USP-2 dissolution test as described in USP <721>. Preferred formulations and dosage forms are those that when tested by the above method result in release of Compound 1 at least 80% being dissolved in the dissolution media within 30 minutes in a sink environment. Examples of acceptable immediate release dissolution is shown in FIGS. 1 and 2.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

The term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms).

The term “pharmaceutically acceptable salts” is meant to include salts of the active compounds which are prepared with relatively nontoxic acids or bases, Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19 or P. Heinrich, Stahl, Camille G. Wemouth, Handbook of Pharmaceutical Salts, 2002. Wiley-VCH).

The neutral form of Compound 1 may be regenerated by contacting the salt with a base or acid and isolating the parent compound in the conventional manner. The parent form of Compound 1 differs from the various salt forms in certain physical properties, such as solubility in polar solvents, but otherwise the salts are equivalent to the parent form of Compound 1 for the purposes of the present disclosure. The Examples of the current disclosure use Compound 1 acetate salt. A person of skill in the art would recognize that in order to administer the same amount of Compound 1 in freebase or in a different salt form, small adjustments in overall amount administered is necessary. For example, 100 mg of Compound 1 as an acetate salt corresponds to about 85 mg of Compound 1 as a free base and about 90 mg of Compound 1 as an chloride salt.

The term “individual” refers to mammals, which includes primates (especially humans), domesticated companion animals (such as dogs, cats, horses, and the like) and livestock (such as cattle, pigs, sheep, and the like), with dosages as described herein. In some embodiments, the term “individual” refers to a human.

### III. Detailed Description of Embodiments

A. Pharmaceutical Formulations

The compositions of the present disclosure comprise Compound 1, a flow aid, a disintegrant, and a lubricant. In addition, other excipients known in the pharmaceutical arts may also be added. Compound 1 can be an amorphous form or a crystalline form. When Compound 1 is a crystalline form, it is preferably an acetate salt form or a chloride salt form. The flow aid is selected from, but not limited to, colloidal silicon dioxide, magnesium trisilicate and calcium phosphate. The disintegrant is selected from, but not limited to, croscarmellose sodium, crospovidone, sodium starch glycolate and carboxymethyl cellulose. The lubricant is selected from, but not limited to, sodium stearyl fumarate and magnesium stearate. Optional additional excipients are preferably selected from brittle fillers such as, but not limited to, mannitol and lactose and ductile fillers such as, but not limited to, microcrystalline cellulose. Formulations of the present disclosure are further described in the following paragraphs.

In some aspects, provided herein are pharmaceutical compositions of Compound 1 or a pharmaceutically acceptable salt thereof. Compound 1 is a compound having the formula

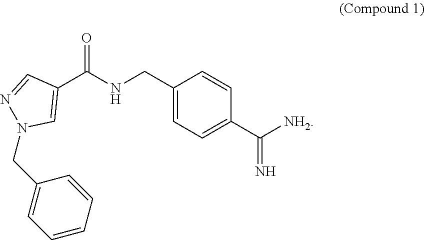 [SMILES: N=C(N)c1ccc(CNC(=O)c2cnn(Cc3ccccc3)c2)cc1]

In some embodiments, a pharmaceutically acceptable salt of Compound 1 corresponds to Formula I.

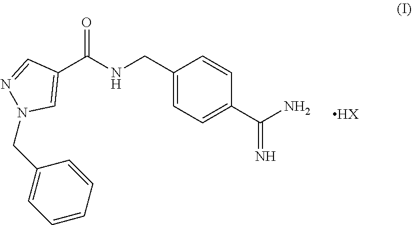

wherein X is a pharmaceutically acceptable anion of a protic acid.

A variety of protic acids are suitable for making a pharmaceutically acceptable salt of Formula I. It can be seen that the pharmaceutically acceptable anion of the protic acid is dependent upon the protic acid used. For example, protic acids useful in the present disclosure include hydrochloric acid, hydrobromic acid, sulfonic acid, tosylic acid (p-toluenesulfonic acid), methanesulfonic acid, nitric acid, or acetic acid. Thus, pharmaceutically acceptable anions of a protic acid include chloride (Cl⁻), bromide (Br⁻), sulfonate (HS(O)₂O⁻), tosylate (TsO⁻), mesylate (MsO⁻), nitrate (NO₃⁻) and acetate (CH₃C(O)O⁻), or combinations thereof.

In some embodiments, the pharmaceutically acceptable anion of a protic acid is acetate, and the pharmaceutically acceptable salt of Formula I is represented by Formula Ia:

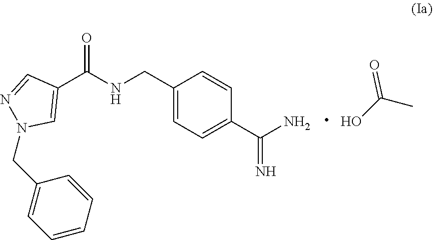

Pharmaceutically acceptable salts of Formula I can be produced using a number of conventional means in the art. For example, the free base form of Compound 1 may be contacted with a stoichiometric amount of the appropriate acid in water, an organic solvent, or a mixture of the two. In some embodiments, pharmaceutically acceptable salts of Formula I are made in nonaqueous media such as an ether, ethyl acetate, ethanol, isopropanol, or acetonitrile. In some embodiments, the pharmaceutically acceptable salts of Formula I are made by dissolving Compound 1 in water, adding a suitable amount of HX to form a mixture, and adding a nonaqueous solvent, such as the nonaqueous media described above to crystallize the salt. In some embodiments, a suitable amount of HX is a stoichiometric amount. It is understood the HX comprises a hydrogen and an X is a pharmaceutically acceptable anion of a protic acid as defined above.

Compound 1 can be formulated and delivered in a variety of forms including amorphous forms and crystalline forms. Amorphous forms of Compound 1 can comprise and be manufactured by any known compositions and methods. Such amorphous forms include those comprised of essentially Compound 1 and those comprised of Compound 1 dispersed in a matrix. Such amorphous forms can be made by thermal processes or solvent processes known in the art. In particular, amorphous forms of Compound 1 disclosed in US patent application US 2021/0009525 published Jan. 14, 2021 are included.

Crystalline forms of Compound 1 can be any of those known. Such forms include neutral forms referred to as free base forms or salt forms. In some embodiments, salt forms of Compound 1 include crystalline acetate and chloride forms. In some embodiments, crystalline salt forms are the acetate forms disclosed in US 2021/0009525 incorporated herein by reference. For example, the crystalline acetate salt forms utilized in the examples are included in such preferred salt forms.

In some aspects, provided herein are pharmaceutical compositions for oral delivery comprising:
a) 1-benzyl-1H-pyrazole-4-carboxylic acid 4-carbamimidoylbenzylamide (Compound 1)

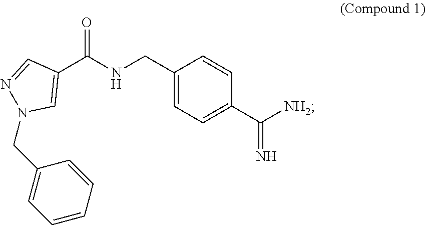 [SMILES: N=C(N)c1ccc(CNC(=O)c2cnn(Cc3ccccc3)c2)cc1]

b) a flow aid;
c) a disintegrant; and
d) a lubricant.

In some embodiments, the pharmaceutical composition further comprises,
e) a brittle filler,
f) a ductile filler and
g) an anti-adherent.

When referring to encapsulated powders and the percent by weight of the compositions, formulations, and solid dosage forms described herein, it is understood that the percent by weight does not include the weight of the capsule.

In some embodiments, the compositions, formulations, and dosage forms described herein comprises about 20 to 70% by weight of Compound 1. In some embodiments, the pharmaceutical composition comprises about 25 to 50% by weight of Compound 1. In some embodiments, the pharmaceutical composition comprises about 25 to 45% by weight of Compound 1. In some embodiments, the pharmaceutical composition comprises about 50% by weight of Compound 1. In some embodiments, the pharmaceutical composition comprises about 33.3% by weight of Compound 1. In some embodiments, the percent by weight of Compound 1 is the amount of Compound 1 in an acetate salt form. In some embodiments the percent by weight of Compound 1 is the amount of Compound 1 in free base form. In some embodiments, Compound 1 in an acetate salt form is anhydrous Crystalline Form I characterized by an X-ray powder diffraction (XRPD) pattern that comprises peaks at 10.0, 18.1, 18.6, 20.1, and 23.9 degrees, ±0.5 degrees, 20, wherein said XRPD is made using Cu Kα radiation. In some embodiments, the XRPD pattern further comprises a peak at 20.5 degrees, +0.5 degrees, 20. In some embodiments, anhydrous Crystalline Form I of Compound 1 is further characterized by a melting point of about 253° C., and an aqueous solubility of about 8.3 mg/mL at 25° C.

In some embodiments, the compositions, formulations, and dosage forms of the present invention include a disintegrant. Disintegrants are a class of pharmaceutical excipients incorporated into pharmaceutical formulations and solid dosage forms. Examples of disintegrants include, but are not limited to, croscarmellose sodium, crospovidone, sodium starch glycolate, carboxymethylcellulose, polyvinylpyrrolidone, methyl cellulose, starch, lower alkyl-substituted hydroxypropyl cellulose, microcrystalline cellulose & powdered cellulose. Certain disintegrants included at the right level, help provide formulations and dosage forms with the benefits described above including good physical and chemical stability and good dissolution properties. In some embodiments, disintegrants are selected from croscarmellose sodium, crospovidone, sodium starch glycolate & carboxymethylcellulose. Carboxymethylcellulose can be the sodium or calcium form. Generally, any product that falls within the scope of the definitions of these materials in the USP or Pharmaceutical Excipient Handbook is acceptable for inclusion. In some embodiments, disintegrants are croscarmellose sodium or crospovidone. In some embodiments, disintegrant is croscarmellose sodium. A single disintegrant is often used but mixtures of more than one disintegrant can also be used. The amount of disintegrant included in the formulation or dosage form can vary substantially as long as the desired properties of the composition are met. In some embodiments, the disintegrant comprises from about 4% to about 20% by weight of the composition. In some embodiments, the disintegrant comprises from about 3% to about 15% by weight of the composition. In some embodiments, the disintegrant comprises from about 5 to about 10% by weight of the composition. In some embodiments, the disintegrant comprises from about 6 to about 8% by weight of the composition. In some embodiments, the disintegrant comprises from about 7.3% by weight of the composition.

In some embodiments, the formulations and dosage forms of the present invention include a flow aid. Flow aids can also be referred to as glidants. The inventors have discovered that the various forms of Compound 1 can have poor flow and can impart this poor flow to mixtures used as formulations or in manufacturing dosage forms. Such poor flow may tend to increase with increasing drug present in the formulation. In particular, crystalline forms of the acetate salt of Compound 1 imparts poor flow to formulations and mixtures used to form the dosage forms of the present invention. Addition of certain flow aids in appropriate amounts have been found to improve flow to the compositions used to form the dosage forms such as encapsulated powders and compressed tablets allowing their efficient manufacture. However, increased amounts of flow aids tend to promote degradation or poor chemical stability of Compound 1 upon storage—particularly when stored in the presence of water. Thus, the present disclosure describes formulations that balance the need for manufacturability while also maintaining the stability and chemical integrity of the active agent: Compound 1.

One indicator of poor chemical stability is an increase in the amount of “The Amide Degradant” present in samples over time. As described above, the addition of a flow aid (e.g., colloidal silicon dioxide in various forms) to formulations or dosage forms of Compound 1 can decrease the chemical stability of the composition as indicated by an increase in the rate that The Amide Degradant increases upon storage. However, a carefully controlled amount of a flow aid (e.g., colloidal silicon dioxide) provides acceptable flow while still having acceptable chemical stability.

Exemplary flow aids include, but are not limited to, cellulose—including microcrystalline and silicified forms, colloidal silicon dioxide, magnesium trisilicate and calcium phosphate. In some embodiments, the addition of about 5% to about 0.25% by weight of a flow aid provides a combination of acceptable flow and acceptable chemical stability. In some embodiments, the addition of about 5% to about 0.5% by weight of a flow aid provides a combination of acceptable flow and acceptable chemical stability. In some embodiments, the compositions disclosed herein include about 0.5 to about 3% flow aid by weight. In some embodiments, the compositions disclosed herein include about 1 to about 3% flow aid by weight. In some embodiments, the formulations and dosage forms of the present invention comprise about 1 to 3% of a flow aid (e.g., colloidal silicon dioxide) by weight. In some embodiments, the compositions disclosed herein include about 1% flow aid by weight. In some embodiments, the formulations and dosage forms of the present invention comprise about 1% of a flow aid (e.g., colloidal silicon dioxide) by weight. In some embodiments, the compositions disclosed herein include about 1.5% flow aid by weight. In some embodiments, the formulations and dosage forms of the present invention comprise about 1.5% of a flow aid (e.g., colloidal silicon dioxide) by weight. In some embodiments, the compositions disclosed herein include about 2% flow aid by weight. In some embodiments, the formulations and dosage forms of the present invention comprise about 2% of a flow aid (e.g., colloidal silicon dioxide) by weight. In some embodiments, the compositions disclosed herein include about 3% flow aid by weight. In some embodiments, the formulations and dosage forms of the present invention comprise about 3% of a flow aid (e.g., colloidal silicon dioxide) by weight.

In some embodiments, the formulations and dosage forms of the present invention include a lubricant. Examples of lubricants include, but are not limited to, sodium stearyl fumarate, magnesium stearate, stearic acid, zinc stearate, sodium lauryl sulfate, magnesium oxide, poloxamer and polyethylene glycol, each of which can be incorporated into the presently disclosed formulation. In some embodiments, the lubricant is sodium stearyl fumarate, or magnesium stearate. The amount of lubricant present in the formulations and dosage forms of the present invention can vary substantially depending on the formulation, process and lubricant chosen. However, in general, the amount of lubricant present in the compositions of the present invention is from about 0.5 to about 10% by weight. In some embodiments, the compositions comprise about 0.5 to about 5.0% lubricant by weight. In some embodiments, the compositions comprise about 0.25 to about 5.0% lubricant by weight. In some embodiments, the compositions comprise about 1.0 to about 3.0% lubricant by weight. In some embodiments, the compositions comprise about 1.0 to about 4.0% lubricant by weight. In some embodiments, the compositions of the present invention comprise about 2.5% sodium stearyl fumarate by weight. In some embodiments, the compositions comprise about 1.5% lubricant by weight.

In some embodiments, the formulations and dosage forms of the present invention include a brittle filler. In some embodiments, a brittle filler is not necessary. Examples of brittle fillers include, but are not limited to, mannitol, lactose, dicalcium phosphate and calcium carbonate. Such excipients can exist in various forms, any of which are included in this invention. For example, “dicalcium phosphate” includes various forms including but not limited to dibasic calcium phosphate anhydrous (DCPA), dibasic dihydrate calcium phosphate, tribasic calcium phosphate & functionalized forms such as Fujicalin (manufactured by Fuji Chemical Industries Company), “calcium carbonate” includes functionalized forms such as Omyapharm. (manufactured by Omya International AG) and “lactose” includes but is not limited to anhydrous and monohydrate forms as well as processed forms such as Fast-Flo Lactose, Modified. (manufactured by Foremost Farms, USA) The compositions of this present invention can include from 0.0 to about 40% by weight of a brittle filler. In some embodiments, the compositions comprise about 5 to about 35% by weight of a brittle filler. In some embodiments, the compositions comprise about 10 to about 30% by weight of a brittle filler. In some embodiments, the compositions comprise about 10 to about 25% by weight of a brittle filler. In some embodiments, the compositions comprise about 5 to about 17.5% by weight of a brittle filler. In some embodiments, the compositions comprise about 7.5 to about 15% by weight of a brittle filler. In some embodiments, the compositions comprise about 15 to about 20% by weight of a brittle filler. In some embodiments, the compositions comprise about 5 to about 11.5% by weight of a brittle filler.

In some embodiments, the formulations and dosage forms of the present invention include a ductile filler. In some embodiments, a ductile filler is not necessary. Examples of ductile fillers include, but are not limited to, microcrystalline cellulose, silicified microcrystalline cellulose, powdered cellulose & starch. Such materials may exist in a variety of forms. For example, microcrystalline cellulose is sold in a wide variety of grades under the brand name Avicel. (Dupont Pharma Solutions). Many of these grades contain additives in addition to microcrystalline cellulose. In general, any of these grades that are approved for use in oral pharmaceutical products may be used as long as the additives do not adversely impact the properties of the compositions. In some embodiments, the ductile filler is microcrystalline cellulose. The compositions of the present invention may contain from 0 to about 50% by weight of a ductile filler. In some embodiments, when the composition contains a ductile filler, it is present in an amount from about 5 to about 45% by weight. In some embodiments, when the composition contains a ductile filler, it is present in an amount from about 15 to about 45% by weight. In some embodiments, when the composition contains a ductile filler, it is present in an amount from about 25 to about 40% by weight. In some embodiments, when the composition contains a ductile filler, it is present in an amount from about 20 to about 37.5% by weight. In some embodiments, when the composition contains a ductile filler, it is present in an amount from about 30 to about 40% by weight. In some embodiments, when the composition contains a ductile filler, it is present in an amount from about 34.4% by weight.

In some embodiments, the formulations and dosage forms of the present invention include an anti-adherent. In some embodiments, an anti-adherent is not necessary. Inclusion of an anti-adherent is often desirable when all or part of the composition is granulated during processing. Examples of anti-adherents include, but are not limited to, talc, corn starch, colloidal silica, DL-leucine, sodium lauryl sulfate, and various stearates. In some embodiments, when an anti-adherent is included in the composition, it is talc. In some embodiments, when the composition contains an anti-adherent, it is present in an amount from about 3 to about 15% by weight. In some embodiments, when the composition contains an anti-adherent, it is present in an amount from about 5 to about 10% by weight. In some embodiments, when the composition contains an anti-adherent, it is present in an amount from about 8% by weight. In some embodiments, when the composition contains an anti-adherent, it is present in an amount from about 5% by weight.

In some embodiments, the composition comprises, about 0.25 to about 5 wt % colloidal silicon dioxide, about 0.5 to about 5% sodium stearyl fumarate and about 4 to about 10% croscarmellose sodium by weight.

In some embodiments, the composition comprises, about 1.5 wt % colloidal silicon dioxide, about 1.5% sodium stearyl fumarate and about 7.3% croscarmellose sodium by weight.

In some embodiments, the composition comprises, about 30-50% of Compound 1; 30-40% of ductile filler; 10-20% brittle filler, about 2-10% anti-adherent; about 4 to about 10% disintegrant; about 0.25 to about 5% flow aid, about 0.5 to about 5% lubricant by weight.

In some embodiments, the composition comprises, about 33.3% of Compound 1; 34.2% of ductile filler; 17.1% brittle filler, about 5% anti-adherent; about 7.3% disintegrant; about 1.5% flow aid, about 1.5% lubricant by weight.

In some embodiments, the composition comprises, about 50% of Compound 1; 23.1% of ductile filler; 11.6% brittle filler, about 5% anti-adherent; about 7.3% disintegrant; about 1.5% flow aid, about 1.5% lubricant by weight.

In some embodiments, the composition comprises, about 34.8% of Compound 1; 30.6% of ductile filler; 15.3% brittle filler, about 8% anti-adherent; about 7.3% disintegrant; about 1.5% flow aid, about 2.5% lubricant by weight.

In some embodiments, the composition comprises, about 34.8% of Compound 1; 34.4% of ductile filler; 11.5% brittle filler, about 8% anti-adherent; about 7.3% disintegrant; about 1.5% flow aid, about 2.5% lubricant by weight.

In some embodiments, the composition comprises, about 30-50% of Compound 1; 30-40% of microcrystalline cellulose; 10-20% mannitol, about 2-10% talc; about 4 to about 10% croscarmellose sodium; about 0.25 to about 5% colloidal silicon dioxide, about 0.5 to about 5% sodium stearyl fumarate by weight.

In some embodiments, the composition comprises, about 33.3% of Compound 1; 34.2% of microcrystalline cellulose; 17.1% mannitol, about 5% talc; about 7.3% croscarmellose sodium; about 1.5% colloidal silicon dioxide, about 1.5% sodium stearyl fumarate.

In some embodiments, the composition comprises, about 50% of Compound 1; 23.1% of microcrystalline cellulose; 11.6% mannitol, about 5% talc; about 7.3% croscarmellose sodium; about 1.5% colloidal silicon dioxide, about 1.5% sodium stearyl fumarate.

In some embodiments, the composition comprises, about 34.8% of Compound 1; 30.6% of microcrystalline cellulose; 15.3% mannitol, about 8% talc; about 7.3% croscarmellose sodium; about 1.5% colloidal silicon dioxide, about 2.5% sodium stearyl fumarate.

In some embodiments, the composition comprises, about 34.8% of Compound 1; 34.4% of microcrystalline cellulose; 11.5% mannitol, about 8% talc; about 7.3% croscarmellose sodium; about 1.5% colloidal silicon dioxide, about 2.5% sodium stearyl fumarate.

B. Solid Dosage Forms

The dosage forms of the present invention are solid dosage forms intended to be dosed orally. Typically, the solid dosage forms are selected from a compressed tablet or encapsulated powder. The dosage forms comprise the formulations described above. The dosage forms of the present disclosure comprise Compound 1, a flow aid, a disintegrant, and a lubricant. In addition, other excipients known in the pharmaceutical arts may also be added. Compound 1 can be an amorphous form or a crystalline form. When Compound 1 is a crystalline form, it is preferably an acetate salt form or a chloride salt form. The flow aid is selected from, but not limited to, colloidal silicon dioxide, magnesium trisilicate and calcium phosphate. The disintegrant is selected from, but not limited to, croscarmellose sodium, crospovidone, sodium starch glycolate and carboxymethyl cellulose. The lubricant is selected from, but not limited to, sodium stearyl fumarate and magnesium stearate. Optional additional excipients are preferably selected from, but not limited to, brittle fillers such as mannitol and lactose and ductile fillers such as, but not limited to, microcrystalline cellulose. Solid dosage forms of the present disclosure are further described in the following paragraphs.

The formulations of the present invention are well suited to manufacture into dosage forms intended for dosing Compound 1 orally to subjects. Therefore, the present disclosure includes solid dosage forms comprising formulations and pharmaceutical compositions of Compound 1 as described herein.

In some embodiments, Compound 1 is an acetate salt of Compound 1. In some embodiments, Compound 1 in an acetate salt form is anhydrous Crystalline Form I characterized by an X-ray powder diffraction (XRPD) pattern that comprises peaks at 10.0, 18.1, 18.6, 20.1, and 23.9 degrees, ±0.5 degrees, 20, wherein said XRPD is made using Cu Kα radiation. In some embodiments, the XRPD pattern further comprises a peak at 20.5 degrees, ±0.5 degrees, 2θ. In some embodiments, anhydrous Crystalline Form I of Compound 1 is further characterized by a melting point of about 253° C., and an aqueous solubility of about 8.3 mg/mL at 25° C.

The dosage forms of the present disclosure can take on a wide range of forms. These include hard and soft dry-filled capsules, compressed compacts, compressed tablets, compressed caplets and similar such forms. The compressed dosage forms can be single layer or multi-layer tablets and they can be coated or uncoated. A particularly preferred dosage form is a compressed form such as a tablet or caplet.

In some embodiments, the solid dosage form is a compressed tablet. The amount of Compound 1 or a pharmaceutically acceptable salt thereof, in a tablet can be about 0.1 to about 500 mg, about 0.1 to about 250 mg, or about 0.1 to about 100 mg. In some embodiments, the amount of Compound 1 present in a tablet is about 10, 25, 50, 100, 200, 300, 400, or 500 mg. In some embodiments, the amount of Compound 1 present in a tablet is about 50, 100, 200, or 400 mg. In some embodiments, the total weight (e.g., active ingredients plus excipients—not including any coating) of the tablet is about 50 to about 1500 mg. For example, the total weight of the solid dosage form is about 100, 150, 200, 300, 400, 500, 600, 700, 800, 900, 1000, or 1500 mg.

In some embodiments, solid dosage forms described herein have certain dissolution properties when dissolved in aqueous dissolution medium. In some embodiments the aqueous dissolution medium is 20 mM sodium phosphate buffered pH 6.8. In some embodiments, the solid dosage forms of the present disclosure are at least 75% dissolved after 5 minutes in aqueous media at 37±0.5° C. in an Apparatus-II (Paddles) with a paddle speed of about 75 rpm. In some embodiments, the solid dosage forms of the present disclosure are at least 85% dissolved after 5 minutes in aqueous media at 37±0.5° C. in an Apparatus-II (Paddles) with a paddle speed of about 75 rpm. In some embodiments, the solid dosage forms of the present disclosure are at least 95% dissolved after 5 minutes in a solution aqueous media at 37±0.5° C. in an Apparatus-II (Paddles) with a paddle speed of about 75 rpm. In some embodiments, the solid dosage forms tested were prepared within a week of the dissolution test. In some embodiments, the solid dosage forms tested were prepared at least a month before performing the dissolution test. In some embodiments, the solid dosage forms tested were prepared at least a three months before performing the dissolution test. In some embodiments, the solid dosage forms tested were prepared at least six months before performing the dissolution test. In some embodiments, the solid dosage forms were incubated for one month at 25° C. with 60% relative humidity (RH) before performing the dissolution test. In some embodiments, the solid dosage forms were incubated for two months at 25° C. with 60% relative humidity (RH) before performing the dissolution test. In some embodiments, the solid dosage forms were incubated for three months at 25° C. with 60% relative humidity (RH) before performing the dissolution test. In some embodiments, the solid dosage forms were incubated for about one month at 40° C. with 75% relative humidity (RH) before performing the dissolution test. In some embodiments, the solid dosage forms were incubated for about three months at 40° C. with 75% relative humidity (RH) before performing the dissolution test. In some embodiments, the solid dosage forms were incubated for six months at 40° C. with 75% relative humidity (RH) before performing the dissolution test. In some embodiments, the solid dosage forms are tablets.

C. Methods of Making Solid Dosage Forms

The formulations and dosage forms of the present invention can generally be processed into a form for oral delivery of Compound 1 to a subject using any of the processes known in the art. The formulations of the present invention can be dosed as powders or suspensions of various types, or formed into solid dosage forms such as beads, encapsulated powders or tablets. In any case, any method known in the art may be used to blend the components of the formulation and modify their density and particle size to facilitate dosing and, when the formulation is formed into a dosage form, to facilitate the manufacture of said dosage form.

Granulation of various types is an optional, but often preferred processing method to utilize. In the case of powdered or particulate formulations, granulation often improves the homogeneity and flow of the blends. In addition, when the formulation is dosed as a reconstituted solution or suspension, granulation can improve the dissolution or dispersion of the material. When the formulation is used to form a solid dosage form, granulation of all or part of the formulation can improve the flow and facilitate the filling of capsules or the manufacture of compressed tablets or caplets. The formulations of the present invention can be granulated by “dry processes” or “mechanical processes” wherein all or part of the formulation is compressed. For example, the material to be granulated may be compressed by “slugging” or roller compaction followed by milling to form granules. Wet granulation techniques can also be employed. Examples of wet granulation include fluid bed and high shear granulation. Granulation is often combined with milling and sieving techniques to obtain granules of the desired size. Granules formed by such techniques generally show improved flow, wetting and dispersion. In the case of forming the solid dosage forms of the present invention, granulation of part or all of the formulation can improve the flow properties of the formulation for filling capsules or for forming compressed tablets.

The Examples section of the current application includes further details regarding the steps used to prepare compositions and formulations of the present disclosure.

D. Methods of Treatment

The methods of treatment of the present invention are generally methods of preventing or treating a subject with a plasma kallikrein-dependent condition or disease comprising orally delivering the formulations or dosage forms described above to a subject in an effective amount of Compound 1 and an effective dosing frequency. Preferably Compound 1 is delivered in the form of a dosage form intended for oral consumption. Methods of treatment of the present disclosure are further described in the following paragraphs.

Accordingly, in some aspects, provided herein are methods of treating plasma kallikrein-dependent diseases or conditions using Compound 1 in tablet formulations of pharmaceutical dosage forms as described herein.

Plasma kallikrein-dependent diseases or conditions, including disorders of blood coagulation, such as thrombosis, and other PK-dependent diseases and conditions. For example, the compounds inhibit the formation of thrombin by the intrinsic pathway and thus reduce the risk of new pathogenic thrombus formation (reocclusion), and also improve fibrinolytic-induced reperfusion when given as adjunctive therapy with a fibrinolytic regimen. Compound 1 is also useful for treating other disease and disorders that are mediated by plasma kallikrein, for example, without limitation, diabetic macular edema, diabetic retinopathy, hereditary angioedema with C1 inhibitor deficiency, acute liver injury, inflammation and anaphylaxis, exacerbation of hemorrhagic transformation and cerebral edema after treatment with recombinant tissue plasminogen activator (tPA), chemical-sensitized renal damage, ischemic stroke, hemorrhagic stroke, hypertension and its vascular complications (including retinopathy and nephropathy), cerebrovascular edema, pulmonary hypertension, inflammation, pain, acute myocardial infarction (MI), deep vein thrombosis (DVT), complications from fibrinolytic treatment (e.g., with tissue plasminogen activator, streptokinase) following stroke or MI, angina, angioedema, sepsis, arthritis, complications of cardiopulmonary bypass, capillary leak syndrome, inflammatory bowel disease, diabetes and its vascular complications (including retinopathy, diabetic macular edema, nephropathy and neuropathy), age-related macular degeneration, retinal vein occlusions, brain edema, ischemia-reperfusion injury, angiogenesis (e.g., in cancer), asthma, anaphylaxis, and cerebrovascular complications of neurological conditions (e.g., Alzheimer's Disease, Parkinson's Disease, multiple sclerosis, CNS infections, and glioblastoma multiforme).

Plasma kallikrein (PK), a serine protease present in plasma as the inactive zymogen precursor plasma prekallikrein (prePK), is proteolytically activated by FXIIa. In a positive feedback loop, PK proteolytically activates the zymogen FXII, leading to additional FXIIa formation, further amplifying its own activation. FXIIa also activates the zymogen FXI to active FXIa, which results in the initiation of the intrinsic (contact) pathway of blood coagulation, resulting in generation of thrombin, and cleavage of fibrinogen. Importantly, PK cleaves high molecular weight kininogen (HMWK) to generate bradykinin. Bradykinin is able to open the tight junctions between endothelial cells lining blood vessels by activating its receptors, B1 and B2, present on the endothelial cells' surface, and thus allowing fluid and plasma protein to extravasate into tissue, a condition known as increased vascular permeability. Disruption of tight junctions of the blood-brain barrier, and consequent leakage of plasma and proteins into the brain (edema) have also been associated with neurodegenerative diseases, such as Alzheimer's Disease, Parkinson's Disease, and multiple sclerosis (MS), as well as with CNS infections and brain tumors. For example, peritumoral brain edema results in poorer prognosis in patients with glioblastoma multiforme (K. Schoenegger et al., Eur J Neurol. (2009) 16(7):874-78). The increased vascular permeability caused by bradykinin formation can result in the accumulation of excess fluid (edema) in many tissues and organs in various diseases, e.g., angioedema, cystoid macular edema, diabetic macular edema, macular edema after retinal vein occlusion, cerebrovascular edema following stroke or head trauma, and capillary leak syndrome. For example, Compound 1 has been shown to reduce retinal vascular permeability in angiotensin-II-treated rodents, as did the BK receptor antagonist Hoe-140 (J. A. Phipps et al., Hypertension (2009) 53:175-81). Activation of prePK and the contact system has also been shown to cause anaphylaxis, e.g., in patients treated with contaminated heparin (T. K. Kishimoto et al., N. Engl. J. Med. (2008) 358:2457-67).

The importance of BK in vasogenic edema is further illustrated in hereditary angioedema, in which individuals have little or no functional Cl-Inhibitor (the major endogenous inhibitor of PK). High levels of bradykinin are generated in these individuals resulting in extravasation of fluid and protein from the plasma into soft tissue, thus causing life-threatening edema. C1-Inhibitor is also known to be involved in the pathogenesis of age-related macular degeneration (S. Ennis et al., Lancet (2008) 372:1828-34) and ischemia-reperfusion injury following organ transplant or myocardial infarction (D. Inderbitzin et al., Eur. Surg. Res. (2004) 36:142-47; G. Horstick et al., Circulation (2001) 104:3125-31). Bradykinin and its receptors have been shown to be involved in tumor angiogenesis (Y. Ikeda et al. Cancer Res (2004) 64:5178-85), pulmonary hypertension (L. Taraseviciene-Stewart et al. Peptides (2005) 26:1292-300), and asthma (P. J. Barnes, “Recent Progress on Kinins”, (1992) AAS38/III, Birkhauser Verlag, Basel).

In patients with angioedema conditions, a small polypeptide PK inhibitor (DX-88, ecallantide) alleviates edema in patients with hereditary angioedema (A. Williams et al., Transfus. Apher. Sci. (2003) 29:255-58; L. Schneider et al., J Allergy Clin Immunol. (2007) 120(2):416-22; J. H. Levy et al., Expert Opin. Invest. Drugs (2006) 15:1077-90). A bradykinin B2 receptor antagonist, icatibant, is also effective in treating hereditary angioedema (K. Bork et al., J. Allergy Clin. Immunol. (2007) 119:1497-503). PK generates bradykinin, therefore inhibition of PK inhibits bradykinin production.

In thrombogenesis resulting from fibrinolytic treatment (e.g., tissue plasminogen activator, streptokinase), higher levels of PK are found in patients undergoing fibrinolysis (H. M. Hoffmeister et al., J. Cardiovasc. Pharmacol. (1998) 31:764-72). Plasmin-mediated activation of the intrinsic pathway has been shown to occur in plasma and blood, and was markedly attenuated in plasma from individuals deficient in any of the intrinsic pathway components (G. A. Ewald et al., Circulation (1995) 91:28-36). Individuals who have had an acute MI were found to have elevated levels of activated PK and thrombin (H. M. Hoffmeister et al., Circulation (1998) 98:2527-33).

Ecallantide reduced brain edema, infarct volume and neurological deficits in an animal model of ischemic stroke (C. Storini et al., J. Pharm. Exp. Ther. (2006) 318:849-54). C1-INH reduced infarct size in a mouse model of middle cerebral artery occlusion (M. G. De Simoni et al., Am. J. Pathol. (2004) 164:1857-63; N. Akita et al., Neurosurg. (2003) 52:395-400). Compound 1 was shown to reduce infarction volume and cerebrovascular edema in a rat model of ischemic stroke, and expansion of intracerebral hemorrhage in a model of hemorrhagic stroke (WO2009/0971). B2 receptor antagonists were found to reduce the infarct volume, brain swelling, and neutrophil accumulation, and were neuroprotective in an animal model of ischemic stroke (S. Zausinger et al., Acta Neurochir. Suppl. (2003) 86:205-07; D. B. Lumenta et al., Brain Res. (2006) 1069:227-34; L. Ding-Zhou et al., Br. J. Pharmacol. (2003) 139:1539-47).

It has been found that prePK levels are higher in diabetics, especially those with proliferative retinopathy, and correlate with fructosamine levels (B.-B. Gao et al., Nature Med. (2007) 13:181-88; K. Kedzierska et al., Archives Med. Res. (2005) 36:539-43). PrePK is also found to be elevated in diabetics and is highest in those with a sensomotor neuropathy (M. Christie et al., Thromb. Haemostas. (1984) 52:221-23). PrePK levels are elevated in diabetics, and are associated with increased blood pressure, independently correlate with the albumin excretion rate, and are elevated in diabetics with macroalbuminuria, suggesting prePK may be a marker for progressive nephropathy (A. A. Jaffa et al., Diabetes (2003) 52:1215-21). B1 receptor antagonists have been found to decrease enhanced vascular permeability and plasma leakage into various organs, including the skin and retina, in rats with streptozotocin-induced diabetes (S. R. Lawson et al., Eur. J. Pharmacol. (2005) 514:69-78; S. R. Lawson et al., Regul Pept. (2005) 124:221-24). B1 receptor antagonists can also prevent streptozotocin-treated mice from developing hyperglycemia and renal dysfunction (A. Zuccollo et al., Can. J. Physiol. Pharmacol. (1996) 74:586-89).

E. Kits

The disclosure also encompasses kits comprising at least one pharmaceutical dosage form as described herein.

In some aspects, provided herein are kits comprising a tablet comprising Compound 1 as described herein. In some embodiments, provided herein are one or more unit dosage tablet described herein.

Some of the kits described herein include a label describing a method of administering pharmaceutical formulations described herein. Some of the kits described herein include a label describing a method of treating a plasma kallikrein dependent disease or condition.

The pharmaceutical dosage forms comprising Compound 1 of the present disclosure can be packaged in a bottle, jar, vial, ampoule, tube, blister pack, or other container-closure system approved by the Food and Drug Administration (FDA) or other regulatory body, which may provide one or more unit dosages containing tablets comprising Compound 1 or a phamectucially acceptable salt thereof. In some embodiments, the tablet comprising Compound 1 is packaged in a bottle. The package or dispenser may also be accompanied by a notice associated with the container in a form prescribed by a governmental agency regulating the manufacture, use, or sale of pharmaceuticals, the notice indicating approval by the agency. In certain aspects, the kit may include a tablet comprising Compound 1 as described herein, a container closure system including the formulation or one or more dosage units form including the formulation, and a notice or instructions describing a method of use as described herein.

### IV. EXAMPLES

The following examples are provided to illustrate, but not limited the current disclosure.

### Example 1: Chemical Stability of Compacts & the Amide Degradant

Compacts of Compound 1 and various tableting excipients were made and placed in temperature- and humidity-controlled environments to determine how the stability of Compound 1 was impacted. The excipients used fall into 5 categories: 1) “Brittle Filler”, 2) “Ductile Filler”, 3) “Disintegrant”, 4) “Lubricant”, and 5) a “Flow Aid.” Each of these five categories of excipients were not used in each compact.

### Manufacture of Compacts

The compacts were made by mixing the components listed in Table 1 in a glass vial using a Turbula T2C blender and then compressing 100 mg of each mixture using 14 inch round, flat-face tooling using a compression force of 1 kN.

Each of the excipients referred to in Table 1 are described below.
1) “Lactose”, commonly referred to as a “brittle filler”, was the monohydrate form manufactured by Sheffield and was within the USP definition of “Lactose Monohydrate.” (See Handbook of Pharmaceutical Excipients (HPE))
2) “Mannitol”, commonly referred to as a “brittle filler”, was manufactured by Millipore and was withing the USP definition of “Mannitol.” (See HPE)
3) “MCC”, commonly referred to as a “ductile filler”, was microcrystalline cellulose manufactured by Dupont and was within the USP definition of “cellulose, microcrystalline.” (See HPE)
4) “CSS”, commonly referred to as a “disintegrant”, was croscarmellose sodium manufactured by FMC and was within the USP definition of “croscarmellose sodium.” (See HPE)
5) “Crospov”, commonly referred to as a “disintegrant”, was crospovidone manufactured by BASF and was within the USP definition of “crospovidone.” (See HPE)
6) “SSF”, commonly referred to as a “lubricant”, was sodium stearyl fumarate manufactured by JRS Pharma and was within the USP definition of “sodium stearyl fumarate.” (See HPE)
7) “Talc”, commonly referred to as a “anti-adherent”, was talc that is a naturally occurring hydropolysilicate mineral manufactured by Barretts and was within the USP definition of “talc.” (See HPE)
8) “SiO2”, commonly referred to as a “flow aid” was colloidal silicon dioxide manufactured by Evonik and was within the USP definition of “colloidal silicon dioxide.” (See HPE)
9) “Syloid”, commonly referred to as a “flow aid” was a proprietary form of colloidal silicon dioxide (Syloid 244FP) manufactured by W. R. Grace and was within the USP definition of “colloidal silicon dioxide.” (See HPE)

### Testing Chemical Stability

The prepared compacts were exposed to 40° C. & 75% RH for 8 weeks. After 8 weeks, the compacts were prepared for purity testing by trituration and dissolving in 4/1 Acetonitrile/water at 0.4 mg/mL final concentration of Compound 1. All undissolved excipients were separated via centrifugation and the supernatant analyzed by reverse phase HPLC. A gradient method using a Zorbax Eclipse XDB-C18, 4.6×50 mm column was used with 0.1% formic acid in water (mobile phase A) and acetonitrile (mobile phase B) as the mobile phases. The gradient program ran from 5% to 80% mobile phase B over 8 minutes. The primary degradant observed had a retention time 1.25-fold that of the active. The structure of this degradant (referred to as “The Amide Degradant”) was determined to be the structure shown below:

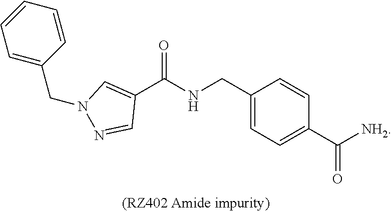 [SMILES: NC(=O)c1ccc(CNC(=O)c2cnn(Cc3ccccc3)c2)cc1]

### Results

Table 1, below, shows the composition of 6 compacts tested as well as the amount of a key degradant referred to as “The Amide Degradant” after exposure of the compacts to 40′ C & 75% RH for 8 weeks. Data for drug alone (“Control”) is also shown.

TABLE 1
Compacts of Compound 1 & Amide Degradant

| Material | Control | Example 1A | Example 1B | Example 1C | Example 1D | Example 1E | Example 1F |
|---|---|---|---|---|---|---|---|
| Compound 1 | 100 | 20 | 20 | 20 | 20 | 20 | 20 |
| Brittle Filler, % | 0 | Mannitol, 30 | Lactose, 30 | Mannitol, 30 | Lactose, 30 | Mannitol, 30 | Mannitol, 30 |
| Ductile Filler, % | 0 | MCC, 35 | MCC, 35 | MCC, 35 | MCC, 35 | MCC, 35 | MCC, 35 |
| Disintegrant, % | 0 | CSS, 10 | Crospov, 10 | 0 | CCS, 10 | Crospov, 10 | 0 |
| Lubricant, % | 0 | SSF, 5 | SSF, 5 | Talc, 15 | 0 | 0 | 0 |
| Flow Aid, % | 0 | 0 | 0 | 0 | SiO2, 5 | SiO2, 5 | Syloid, 15 |
| The Amide | 0.34 | 0.32 | 0.49 | 0.55 | 0.96 | 1.51 | 3.63 |
| Degradant, % | | | | | | | |

First note in Table 1 that the amount of the primary degradant, “The Amide Degradant”, was highest for 1F which was composed of 1500 of Syloid, a proprietary Flow Aid composed of colloidal silicon oxide. Also, the 1D and 1E compacts had the next most degradant formed. Each of these compacts contained the Flow Aid, colloid silicon oxide (a non-proprietary version). However, the addition of colloidal silicon oxide to the excipient and Compound 1 blends greatly improved their flow properties.

Thus, these results indicated that despite some formation of undesirable Amide Degradant, the addition of silicon oxide was needed to have good flow to make tablet manufacture practical.

### Example 2: Select Tablets

The following example provides a description of how select tablets were prepared.

Tablets 1 and 2 were manufactured by blending Compound 1 with the excipients with the exception of sodium stearyl fumarate (excipients are listed in the table for Tablets 1 and 2-see the table below), sieved through a #25 mesh screen, and tumble blended (Turbula T2F, 15 minutes at 49 rpm). Sodium stearyl fumarate was then sieved through a #25 mesh screen and added to blend, tumble blended (Turbula T2F, 2 minutes at 49 rpm), and direct compressed (Natoli RD10A, 1.9-2.1 MPa tensile strength; 7.20 mm×14.60 mm Mod Oval and 8.00 mm×16.50 mm Mod Oval; Natoli HOB #s 182421 and 190767, respectively).

Tablets 3 and 4 were manufactured by blending Compound 1 with the excipients listed in the intra-granular portion of Tablets 3 and 4 (excipients are listed in the table for Tablets 3 and 4—see the table below) to generate an intra-granular material, which composed a total of 92.17% of the final mass of the intra-granular formulation. Raw materials were pre-mixed in a Vanguard Lab Interchangeable V-blender with V-Shell, with a set rotation rate of 10 RPM for up to 30 minutes. The main blend was executed using a Quadro SLS Mill, at a set rotation rate of 10 RPM for up to 30 minutes. Dry granulation by roller compaction and milling was executed using a Gerteis Mini-Polygran (conditions are available if needed). Final granules were then blended with a corresponding extra-granular blend listed in Tablets 3 and 4 (Tables below) by using a Vanguard Lab Interchangeable V-blender with V-Shell, at a set rotation rate of 10 RPM for up to 30 minutes. The process in which these activities are performed generated a common formulation to be used for downstream activities.

To achieve a 50 mg Compound 1 tablet, 175 mg of common granulation is compacted into the designated tooling. To achieve a 200 mg Compound 1 tablet, 700 mg of common granulation is compacted into the designated tooling.

A mass of Compound 1 Common Granulation was filled into the gravity feeder of a Korsch XL 100 tablet press. (The tablet press running conditions for Compound 1 strengths are available as needed.) Several in-process checks were implemented during the tablet compression operation. While the tablet compression was being performed there was routine analysis of manual tablet weight (±5%), thickness, hardness, and friability checks to ensure each met the operation criteria. Tablet thickness and hardness was assessed using a SOTAX ST50. Friability assessments were following guidelines set forth in USP <1216>. A 100% weight sort was performed on collected tablets using a SADE-P4 sorter using the same target limits used during the In-Process check. A 5% weight float was implemented for this procedure to ensure tablet strengths were within the appropriate strength range.

The effect of formulation composition on disintegration and dissolution was examined for tablets and capsules containing Compound 1. Immediate release prototype tablets containing 33% and 50% of Compound 1 and a capsule containing 50% of Compound 1 were tested in a USP apparatus 2 dissolution test further described in Example 4.

Tablet 1

| Component | Grade (Mfr) | Function | Amount (%)* |
|---|---|---|---|
| Compound 1 Acetate Salt | N/A | Active | 33.3 |
| Microcrystalline Cellulose | Avicel PH 101 (Dupont) | Ductile Filler | 34.2 |
| Mannitol | Parteck M100 (Millipore) | Brittle Filler | 17.1 |
| Talc | MP 6030USP (Barretts) | Anti-Adherent | 5.0 |
| Croscarmellose Sodium | Ac-Di-Sol (FMC) | Disintegrant | 7.3 |
| Colloidal Silicon Dioxide | Aerosil 200 Pharma (Evonik) | Flow Aid | 1.5 |
| Sodium Stearyl Fumarate | PRUV (JRS Pharma) | Lubricant | 1.5 |

*Compound 1 salt factor: 0.82 - note that the % amount includes both the active and acetate counterion
Tablet 1 is 167 mg active and 617 mg total mass

Tablet 2

| Component | Grade (Mfr) | Function | Amount (%)* |
|---|---|---|---|
| Compound 1 Acetate Salt | N/A | Active | 50.0 |
| Microcrystalline Cellulose | Avicel PH 101 (Dupont) | Ductile Filler | 23.1 |
| Mannitol | Parteck M100 (Millipore) | Brittle Filler | 11.6 |
| Talc | MP 6030USP (Barretts) | Anti-Adherent | 5.0 |
| Croscarmellose Sodium | Ac-Di-Sol (FMC) | Disintegrant | 7.3 |
| Colloidal Silicon Dioxide | Aerosil 200 Pharma (Evonik) | Flow Aid | 1.5 |
| Sodium Stearyl Fumarate | PRUV (JRS Pharma) | Lubricant | 1.5 |

*Compound 1 salt factor: 0.82 - note that the % amount includes both the active and acetate counterion
Tablet 2 is 167 mg active and 407 mg total mass

Tablet 3

| | | | |
|---|---|---|---|
| Component | Grade (Mfr) | Function | Amount (%)* |

Intragranular Blend

| | | | |
|---|---|---|---|
| Compound 1 Acetate Salt | N/A | Active | 34.8 |
| Microcrystalline Cellulose | Avicel PH 101 (Dupont) | Ductile Filler | 30.6 |
| Mannitol | Parteck M100 (Millipore) | Brittle Filler | 15.3 |
| Talc | MP 6030USP (Barretts) | Anti-Adherent | 5.0 |
| Croscarmellose Sodium | Ac-Di-Sol (FMC) | Disintegrant | 5.0 |
| Colloidal Silicon Dioxide | Aerosil 200 Pharma (Evonik) | Flow Aid | 1.0 |
| Sodium Stearyl Fumarate | PRUV (JRS Pharma) | Lubricant | 0.5 |

Extragranular Blend

| | | | |
|---|---|---|---|
| Talc | MP 6030USP (Barretts) | Anti-Adherent | 3.0 |
| Croscarmellose Sodium | Ac-Di-Sol (FMC) | Disintegrant | 2.3 |
| Colloidal Silicon Dioxide | Aerosil 200 Pharma (Evonik) | Flow Aid | 0.5 |
| Sodium Stearyl Fumarate | PRUV (JRS Pharma) | Lubricant | 2.0 |

*Compound 1 salt factor: 0.82 - note that the % amount includes both the active and acetate counterion
Tablet 3 is 200 mg active and 700 mg total mass

Tablet 4

| | | | |
|---|---|---|---|
| Component | Grade (Mfr) | Function | Amount (%)* |

Intragranular Blend

| | | | |
|---|---|---|---|
| Compound 1 Acetate Salt | N/A | Active | 34.8 |
| Microcrystalline Cellulose | Avicel PH 101 (Dupont) | Ductile Filler | 34.4 |
| Mannitol | Parteck M100 (Millipore) | Brittle Filler | 11.5 |
| Talc | MP 6030USP (Barretts) | Anti-Adherent | 5.0 |
| Croscarmellose Sodium | Ac-Di-Sol (FMC) | Disintegrant | 5.0 |
| Colloidal Silicon Dioxide | Aerosil 200 Pharma (Evonik) | Flow Aid | 1.0 |
| Sodium Stearyl Fumarate | PRUV (JRS Pharma) | Lubricant | 0.5 |

Extragranular Blend

| | | | |
|---|---|---|---|
| Talc | MP 6030USP (Barretts) | Anti-Adherent | 3.0 |
| Croscarmellose Sodium | Ac-Di-Sol (FMC) | Disintegrant | 2.3 |
| Colloidal Silicon Dioxide | Aerosil 200 Pharma (Evonik) | Flow Aid | 0.5 |
| Sodium Stearyl Fumarate | PRUV (JRS Pharma) | Lubricant | 2.0 |

*Compound 1 salt factor: 0.82 - note that the % amount includes both the active and acetate counterion
Tablet 4 is 200 mg active and 700 mg total mass

### Example 3: Select Encapsulated Powders

Raw materials were pre-mixed in a Vanguard Lab Interchangeable V-blender with V-Shell, with a set rotation rate of 10 RPM for up to 30 minutes. The blended powder was manually filled into Size #0 capsules using a Torpac ProFunnel single station capsule filler. These capsules were prepared to be comparators to the tablets for the purpose ofunderstanding in vitro dissolution.

Capsule 1

| | | | Amount |
|---|---|---|---|
| Component | Grade (Mfr) | Function | (%)* |
| Capsule, Hypromellose, #0 white | Qualicaps (Quali-V) | Unit Dosing | one unit |
| Compound 1 Acetate Salt | N/A | Active | 50.0 |
| Microcrystalline Cellulose | Avicel PH 101 (Dupont) | Ductile Filler | 23.1 |
| Mannitol | Parteck M100 (Millipore) | Brittle Filler | 11.6 |
| Talc | MP 6030USP (Barretts) | Anti-Adherent | 5.0 |
| Croscarmellose Sodium | Ac-Di-Sol (FMC) | Disintegrant | 7.3 |
| Colloidal Silicon Dioxide | Aerosil 200 Pharma (Evonik) | Flow Aid | 1.5 |
| Sodium Stearyl Fumarate | PRUV (JRS Pharma) | Lubricant | 1.5 |

*Compound 1 salt factor: 0.82 - note that the % amount includes both the active and acetate counterion
Capsule 1 is 167 mg active and contains 407 mg total mass powder in a single unit

Capsule 2

| | | | Amount |
|---|---|---|---|
| Component | Grade (Mfr) | Function | (%)* |
| Capsule, Hypromellose, #0 white | Qualicaps (Quali-V) | Unit Dosing | one unit |

Intragranular Blend

| | | | |
|---|---|---|---|
| Compound 1 Acetate Salt | N/A | Active | 61.0 |
| Microcrystalline Cellulose | Avicel PH 101 (Dupont) | Ductile Filler | 11.4 |
| Pre-gelatinized Starch | Starch 1500 (Colorcon) | Disintegrant | 5.7 |
| Croscarmellose Sodium | Ac-Di-Sol (FMC) | Disintegrant | 5.0 |
| Colloidal Silicon Dioxide | Aerosil 200 Pharma (Evonik) | Flow Aid | 1.0 |
| Sodium Stearyl Fumarate | PRUV (JRS Pharma) | Lubricant | 1.0 |

Extragranular Blend

| | | | |
|---|---|---|---|
| Microcrystalline Cellulose | Avicel PH 101 (Dupont) | Ductile Filler | 12.5 |
| Croscarmellose Sodium | Ac-Di-Sol (FMC) | Disintegrant | 2.5 |

*Compound 1 salt factor: 0.82 - note that the % amount includes both the active and acetate counterion
Capsule 2 is 200 mg active and contains 400 mg total mass powder in a single unit

### Example 4: Dissolution Testing of the Tablets & Capsules in Example 2 and Example 3

Dissolution was performed per USP <711>. Both 50% and 33% active loading tablets are disintegrated within minutes and dissolved within 5 minutes. Added a graph of the dissolved formulation to the text. Comparatively, encapsulated versions of the formulations would not “release,” and materials remained in the capsule forming a gum-like mass that never fully dissolved. Compare, FIG. 1 & FIG. 2.

### Example 5: Dissolution Consistency and Stability of a Tablet in Example 2 Under Accelerated Conditions

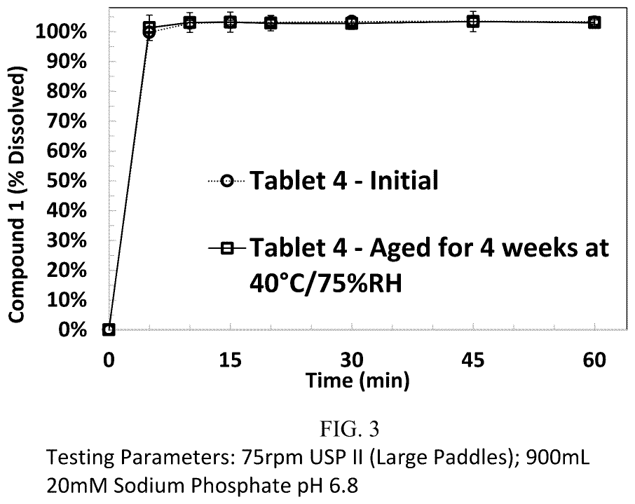

Dissolution was performed per USP <711> on Tablet 4 both before and after aging in a stability chamber for 4 weeks. Tablet 4 initially disintegrates within minutes and dissolved within 5 minutes. This tablet was also aged in a stability chamber at 40° C. and 75% RH for 4 weeks open to the environment. The aged tablet also disintegrates within minutes and dissolved within 5 minutes. Comparison between the initial Tablet 4 and Tablet 4 aged for 40° C. and 75% RH is shown in FIG. 3

Amide degradant formulation was also evaluated on Tablet 4 as a function of time while incubated at 40° C. and 75% RH after 2, 4, and 13 weeks. The results indicated only slight presence of the amide degradant by 13 weeks indicated in the table below.

| Sample | Timepoint | Condition | Amide Degradant, % |
|---|---|---|---|
| Tablet 4 | Initial | | 0.17 |
| | 2 weeks | 40° C./75% RH | 0.19 |
| | 4 weeks | | 0.14 |
| | 13 weeks | | 0.26 |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A pharmaceutical composition for oral delivery comprising
a) 1-benzyl-1H-pyrazole-4-carboxylic acid 4-carbamimidoylbenzylamide (Compound 1) 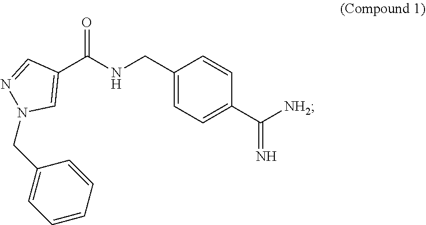 [SMILES: N=C(N)c1ccc(CNC(=O)c2cnn(Cc3ccccc3)c2)cc1]
b) a flow aid;
c) a disintegrant; and
d) a lubricant;
wherein the form of Compound 1 is an amorphous form or a crystalline salt form.

2. The pharmaceutical composition of claim 1, wherein said form of Compound 1 is a crystalline salt form selected from an acetate salt form or a chloride salt form.

3. The pharmaceutical composition of claim 1, wherein said form of Compound 1 is an acetate salt form.

4. The pharmaceutical composition of claim 3, wherein said form of Compound 1 is anhydrous Crystalline Form I characterized by an X-ray powder diffraction (XRPD) pattern that comprises peaks at 10.0, 18.1, 18.6, 20.1, and 23.9 degrees, ±0.5 degrees, 2θ, wherein said XRPD is made using Cu Kα radiation.

5. The pharmaceutical composition of claim 4, wherein Crystalline Form I is substantially free of other polymorphic forms of Compound 1.

6. The pharmaceutical composition of any one of claims 1 to 5, wherein the amount of Compound 1 in said pharmaceutical composition comprises 20% to 70% by weight.

7. The pharmaceutical composition of any one of claims 1 to 5, wherein the amount of Compound 1 in said pharmaceutical composition comprises 25% to 50% by weight.

8. The pharmaceutical composition of any one of claims 1 to 5, wherein the amount of Compound 1 in said pharmaceutical composition comprises at least 25% to 45% by weight.

9. The pharmaceutical composition of any one of claims 1 to 5, wherein the amount of Compound 1 in said pharmaceutical composition comprises about 50% by weight.

10. The pharmaceutical composition of any one of claims 1 to 5, wherein the amount of Compound 1 in said pharmaceutical composition comprises about 33.3% by weight.

11. The pharmaceutical composition of any one of claims 1 to 5, wherein the amount of Compound 1 in said pharmaceutical composition comprises about 34.8% by weight.

12. The pharmaceutical composition of any one of claims 1 to 11, wherein the flow aid is selected from colloidal silicon dioxide, magnesium trisilicate and calcium phosphate.

13. The pharmaceutical composition of claim 12, wherein the flow aid is colloidal silicon dioxide.

14. The pharmaceutical composition of any one of claims 1 to 13, wherein the pharmaceutical composition comprises about 0.25% to about 5% of the flow aid by weight.

15. The pharmaceutical composition of claim 14, wherein the pharmaceutical composition comprises about 0.5% to about 3% of the flow aid by weight.

16. The pharmaceutical composition of claim 14, wherein the pharmaceutical composition comprises about 1.5% of the flow aid by weight.

17. The pharmaceutical composition of any one of claims 1 to 16, wherein the disintegrant is selected from croscarmellose sodium, crospovidone, sodium starch glycolate and carboxymethylcellulose.

18. The pharmaceutical composition of claim 17, wherein the disintegrant is selected from croscarmellose sodium and crospovidone.

19. The pharmaceutical composition of any one of claims 1 to 18, wherein the pharmaceutical composition comprises about 3% to about 15% of the disintegrant by weight.

20. The pharmaceutical composition of claim 19, wherein the composition pharmaceutical comprises about 5% to about 10% of the disintegrant by weight.

21. The pharmaceutical composition of claim 19, wherein the pharmaceutical composition comprises about 7.3% of the disintegrant by weight.

22. The pharmaceutical composition of any one of claims 1 to 21, wherein the lubricant is selected from sodium stearyl fumarate and magnesium stearate.

23. The pharmaceutical composition of claim 22, wherein the lubricant is sodium stearyl fumarate.

24. The pharmaceutical composition of any one of claims 1 to 18, wherein the pharmaceutical composition comprises about 0.25% to 5% of the lubricant by weight.

25. The pharmaceutical composition of claim 19, wherein the composition pharmaceutical comprises about 1% to 4% of the lubricant by weight.

26. The pharmaceutical composition of claim 19, wherein the pharmaceutical composition comprises about 2.5% of the lubricant by weight.

27. The pharmaceutical composition of any one of claims 1 to 11, 14 to 16, 19 to 21, 24 to 26, wherein the flow aid is colloidal silicon dioxide, the lubricant is sodium stearyl fumarate and the disintegrant is croscarmellose sodium.

28. The pharmaceutical composition of any one of claims 1 to 27 wherein the composition further comprises one or more of:
e) a brittle filler,
f) a ductile filler and
g) an anti-adherent.

29. The pharmaceutical composition of claim 28, wherein the brittle filler is selected from mannitol, lactose, dicalcium phosphate and calcium carbonate.

30. The pharmaceutical composition of claim 29, wherein the brittle filler is mannitol.

31. The pharmaceutical composition of any one of claims 28 to 30, wherein the pharmaceutical composition comprises about 5% to about 17.5% of the brittle filler by weight.

32. The pharmaceutical composition of claim 31, wherein the pharmaceutical composition comprises about 7.5% to about 15% of the brittle filler by weight.

33. The pharmaceutical composition of claim 31, wherein the pharmaceutical composition comprises about 11.5% of the brittle filler by weight.

34. The pharmaceutical composition of any one of claims 28 to 33, wherein the ductile filler is selected from microcrystalline cellulose, silicified microcrystalline cellulose, powdered cellulose and starch.

35. The pharmaceutical composition of claim 34, wherein the ductile filler is microcrystalline cellulose.

36. The pharmaceutical composition of any one of claims 28 to 35, wherein the pharmaceutical composition comprises about 15% to 45% of the ductile filler by weight.

37. The pharmaceutical composition of claim 36, wherein the pharmaceutical composition comprises about 20% to 37.5% of the ductile filler by weight.

38. The pharmaceutical composition of claim 36, wherein the pharmaceutical composition comprises about 34.4% of the ductile filler by weight.

39. The pharmaceutical composition of any one of claims 28 to 38, wherein the anti-adherent is selected from talc, corn starch, colloidal silica, DL-leucine, sodium lauryl sulfate, and various stearates.

40. The pharmaceutical composition of claim 39, wherein the anti-adherent is talc.

41. The pharmaceutical composition of any one of claims 28 to 40, wherein the pharmaceutical composition comprises about 3% to 15% of the anti-adherent by weight.

42. The pharmaceutical composition of claim 41, wherein the pharmaceutical composition comprises about 5% to 10% of the anti-adherent by weight.

43. The pharmaceutical composition of claim 41, wherein the pharmaceutical composition comprises about 8% of the anti-adherent by weight.

44. A solid dosage form for oral delivery comprising
a) 1-benzyl-1H-pyrazole-4-carboxylic acid 4-carbamimidoylbenzylamide (Compound 1) 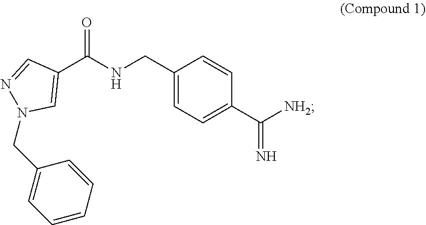 [SMILES: N=C(N)c1ccc(CNC(=O)c2cnn(Cc3ccccc3)c2)cc1]
b) a flow aid
c) a disintegrant, and
d) a lubricant
wherein, the form of Compound 1 is selected from an amorphous form and a crystalline salt form.

45. The solid dosage form of claim 44, wherein the solid dosage form is selected from a compressed tablet, a powder, and a dry-filled capsule.

46. The solid dosage form of claim 45, wherein the solid dosage form is a compressed tablet.

47. The solid dosage form of any one of claims 44 to 46, wherein said form of Compound 1 is a crystalline salt form selected from an acetate salt form or a chloride salt form.

48. The solid dosage form of any one of claims 44 to 46, wherein said form of Compound 1 is an acetate salt form.

49. The solid dosage form of claim 48, wherein said form of Compound 1 is anhydrous Crystalline Form I characterized by an X-ray powder diffraction (XRPD) pattern that comprises peaks at 10.0, 18.1, 18.6, 20.1, and 23.9 degrees, +0.5 degrees, 2θ, wherein said XRPD is made using Cu Kα radiation.

50. The solid dosage form of claim 49, wherein Crystalline Form I is substantially free of other polymorphic forms of Compound 1.

51. The solid dosage form of any one of claims 44 to 50, wherein the amount of Compound 1 in said pharmaceutical composition comprises 20% to 70% by weight.

52. The solid dosage form of any one of claims 44 to 50, wherein the amount of Compound 1 in said pharmaceutical composition comprises 25% to 50% by weight.

53. The solid dosage form of any one of claims 44 to 50, wherein the amount of Compound 1 in said pharmaceutical composition comprises at least 25% to 45% by weight.

54. The solid dosage form of any one of claims 44 to 50, wherein the amount of Compound 1 in said pharmaceutical composition comprises about 50% by weight.

55. The solid dosage form of any one of claims 44 to 50, wherein the amount of Compound 1 in said pharmaceutical composition comprises about 33.3% by weight.

56. The solid dosage form of any one of claims 44 to 50, wherein the amount of Compound 1 in said pharmaceutical composition comprises about 34.8% by weight.

57. The solid dosage form of any one of claims 44 to 56, wherein the flow aid is selected from colloidal silicon dioxide, magnesium trisilicate and calcium phosphate.

58. The solid dosage form of claim 57, wherein the flow aid is colloidal silicon dioxide.

59. The solid dosage form of any one of claims 44 to 58, wherein the pharmaceutical composition comprises about 0.25% to about 5% of the flow aid by weight.

60. The solid dosage form of claim 59, wherein the pharmaceutical composition comprises about 0.5% to about 3% of the flow aid by weight.

61. The solid dosage form of claim 59, wherein the pharmaceutical composition comprises about 1.5% of the flow aid by weight.

62. The solid dosage form of any one of claims 44 to 61, wherein the disintegrant is selected from croscarmellose sodium, crospovidone, sodium starch glycolate and carboxymethylcellulose.

63. The solid dosage form of claim 62, wherein the disintegrant is selected from croscarmellose sodium and crospovidone.

64. The solid dosage form of any one of claims 44 to 63, wherein the pharmaceutical composition comprises about 3% to about 15% of the disintegrant by weight.

65. The solid dosage form of claim 64, wherein the composition pharmaceutical comprises about 5% to about 10% of the disintegrant by weight.

66. The solid dosage form of claim 64, wherein the pharmaceutical composition comprises about 7.3% of the disintegrant by weight.

67. The solid dosage form of any one of claims 44 to 66, wherein the lubricant is selected from sodium stearyl fumarate and magnesium stearate.

68. The solid dosage form of claim 67, wherein the lubricant is sodium stearyl fumarate.

69. The solid dosage form of any one of claims 44 to 68, wherein the pharmaceutical composition comprises about 0.25% to 5% of the lubricant by weight.

70. The solid dosage form of claim 69, wherein the pharmaceutical composition comprises about 1% to 4% of the lubricant by weight.

71. The solid dosage form of claim 70, wherein the pharmaceutical composition comprises about 2.5% of the lubricant by weight.

72. The solid dosage form of any one of claims 44 to 56, 59 to 61, 64 to 66, 69 to 71, wherein the flow aid is colloidal silicon dioxide, the lubricant is sodium stearyl fumarate and the disintegrant is croscarmellose sodium.

73. The solid dosage form of any one of claims 44 to 72 wherein the composition further comprises one or more of:
e) a brittle filler,
f) a ductile filler and
g) an anti-adherent.

74. The solid dosage form of claim 73, wherein the brittle filler is selected from mannitol, lactose, dicalcium phosphate and calcium carbonate.

75. The solid dosage form of claim 74, wherein the brittle filler is mannitol.

76. The solid dosage form of any one of claims 73 to 75, wherein the pharmaceutical composition comprises about 5% to about 17.5% of the brittle filler by weight.

77. The solid dosage form of claim 76, wherein the pharmaceutical composition comprises about 7.5% to about 15% of the brittle filler by weight.

78. The solid dosage form of claim 76, wherein the pharmaceutical composition comprises about 11.5% of the brittle filler by weight.

79. The solid dosage form of any one of claims 73 to 78, wherein the ductile filler is selected from microcrystalline cellulose, silicified microcrystalline cellulose, powdered cellulose and starch.

80. The solid dosage form of claim 79, wherein the ductile filler is microcrystalline cellulose.

81. The solid dosage form of any one of claims 73 to 80, wherein the pharmaceutical composition comprises about 15% to 45% of the ductile filler by weight.

82. The solid dosage form of claim 81, wherein the pharmaceutical composition comprises about 20% to 37.5% of the ductile filler by weight.

83. The solid dosage form of claim 81, wherein the pharmaceutical composition comprises about 34.4% of the ductile filler by weight.

84. The solid dosage form of any one of claims 73 to 83, wherein the anti-adherent is selected from talc, corn starch, colloidal silica, DL-leucine, sodium lauryl sulfate, and various stearates.

85. The solid dosage form of claim 84, wherein the anti-adherent is talc.

86. The solid dosage form of any one of claims 73 to 85, wherein the pharmaceutical composition comprises about 3% to 15% of the anti-adherent by weight.

87. The solid dosage form of claim 86, wherein the pharmaceutical composition comprises about 5% to 10% of the anti-adherent by weight.

88. The solid dosage form of claim 86, wherein the pharmaceutical composition comprises about 8% of the anti-adherent by weight.

89. The solid dosage form of any one of claims 44 to 88, wherein the solid dosage form is at least 80% dissolved after performance of a dissolution test in aqueous dissolution medium solution using USP Apparatus-II (Paddles) with a paddle speed of about 75 rpm for 30 minutes.

90. The solid dosage form of claim 89, wherein the solid dosage form is a tablet, and the tablet was prepared at least 1 month before performing the dissolution test.

91. A method of preventing or treating a subject with a plasma kallikrein-dependent condition or disease comprising orally delivering an effective amount of the pharmaceutical composition of any one of claims 1 to 43 or the solid dosage form of any one of claims 44 to 90.

92. The method of claim 91, wherein the plasma kallikrein-dependent disease or condition is selected from the group consisting of:
diabetic macular edema, diabetic retinopathy, hereditary angioedema with C1 inhibitor deficiency, acute liver injury, inflammation, anaphylaxis, chemical-sensitized renal damage, ischemic stroke, hemorrhagic stroke, hypertension, vascular complications of hypertension, retinopathy, nephropathy, cerebrovascular edema, pulmonary hypertension, inflammation, pain, acute myocardial infarction, deep vein thrombosis, complications from fibrinolytic treatment, angina, angioedema, sepsis, arthritis, complications of cardiopulmonary bypass surgery, capillary leak syndrome, inflammatory bowel disease, diabetes, diabetic retinopathy, diabetic macular edema, diabetic nephropathy, diabetic neuropathy, age-related macular degeneration, retinal vein occlusions, brain edema, ischemia-reperfusion injury, cancer-related angiogenesis, asthma, anaphylaxis, and cerebrovascular complications of Alzheimer's Disease, Parkinson's Disease, multiple sclerosis, Central Nervous System infections, and glioblastoma multiforme.

93. A pharmaceutical composition of any one of claims 1 to 43 or the solid dosage form of any one of claims 44 to 90, for use in the treatment of a plasma kallikrein-dependent disease or condition.

94. The use of a pharmaceutical composition of any one of claims 1 to 43 or the solid dosage form of any one of claims 44 to 90, for the manufacture of a medicament for the treatment of a plasma kallikrein-dependent disease or condition.
